(19) United States
(12) Patent Application Publication
(10) Pub. No.: US 2014/0083886 A1
(43) Pub. Date: Mar. 27, 2014

(54) MODULAR BRACKETS AND INSTRUMENTATION CASE

(71) Applicants: Jason Michael Winterrowd, Columbia City, IN (US); Ralph Brent Sweet, II, Warsaw, IN (US)

(72) Inventors: Jason Michael Winterrowd, Columbia City, IN (US); Ralph Brent Sweet, II, Warsaw, IN (US)

(73) Assignee: Zimmer, Inc., Warsaw, IN (US)

(21) Appl. No.: 13/624,184

(22) Filed: Sep. 21, 2012

(51) Int. Cl. A61B 19/00; A61B 19/02

(52) U.S. Cl. 206/370; 248/542; 206/363

(57) ABSTRACT

A modular bracket and associated instrumentation case configuration is disclosed herein. The modular bracket may include a support member structured to receive a specific instrument and hold the instrument at a fixed position, and an attachment member to facilitate a releasable attachment to a mounting surface such as an instrument tray platform. The modular bracket is shaped to provide an indication of the instrument, to allow user identification of the instrument received by the support member. For example, the indication of the instrument may be provided in the modular bracket by the perimeter shape of the attachment member that resembles an outline of the instrument. A modular instrument case assembly including a tray and housing may provide a platform for modular placement of one or more modular brackets into a specific arrangement of instrumentation.

### BACKGROUND

During medical procedures, various reusable tools and instruments are used by medical professionals to assist with the procedures. For example, in hip replacement or knee replacement surgeries, instruments of a variety of shape and sizes such as blades, saws, drill bits, guides, and the like may be used to shape the bone and establish an area for the insertion of a joint replacement implant. Such instruments are often placed and stored in a storage case. The storage case can be designed to secure tools and instruments in a fixed location for transport and washing, and can have removable trays or other portions that are used to ensure correct exposure to an automated washing machine or sterilization process. Once the instruments are cleaned, the storage case can be used in conjunction with a filtered sterilization container or sterile wrap to provide a sterile transport and protection for the tools.

Existing storage cases often provide brackets or other holding structure to hold a specific set of tools in place, with the set of specific tools being selected or arranged based on the medical procedure. Medical professionals may wish to remove, add, or substitute tools to a storage case, based on doctor preferences or specific medical procedure needs. Without a bracket or other holding structure to secure the tool in the storage case, the tool will remain loose and is more likely to become lost, misplaced, misidentified, or not exposed to a full cleaning. Although some storage cases provide the ability to insert generic brackets, such brackets do not provide a secure fit or identification for most instrumentation shapes.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A illustrates an instrumentation case assembly including an instrumentation tray providing a configuration of modular brackets for receiving multiple medical instruments according to an example;

FIG. 1B illustrates a perspective view of an instrument tray providing a configuration of modular brackets for receiving multiple medical instruments according to an example;

FIG. 2A illustrates a top view of modular brackets placed within an instrument tray to receive various medical instruments according to an example;

FIG. 2B illustrates a top view of modular brackets placed within an instrument tray to receive various medical instruments and placement of a medical instrument using the modular brackets according to an example;

FIGS. 3A and 3B illustrate close-up perspective views of modular brackets to provide engageable mating with respective medical instruments according to an example; and

FIG. 4 illustrates a close-up perspective view of a fastener coupling between a modular bracket and an instrument tray platform according to an example.

### DETAILED DESCRIPTION

Configurations and methods of use for an instrument storage case, modular brackets, and associated storage and receiving structures in an instrument case assembly are generally disclosed herein. In one example, a modular bracket is shaped to provide an indication of a particular instrument or instrument piece that can be received by the modular bracket. This indication can be provided by the shape, color, or other characteristics of the modular bracket. For example, an outline of the modular bracket can be shaped to resemble the specific instrument or instrument piece received by the modular bracket. The instrument received by the modular bracket can be a disposable, temporary, or reusable medical instrument provided in an instrument set (for example, a set of tools associated with a specific medical procedure, medical condition, task, or usage scenario, such as a set of orthopedic (orthopaedic) tools). A plurality of modular brackets can be arranged adjacent to each other in an instrument storage assembly to provide a collection of instruments for a procedure or use case (including cleaning, storage, or transport of the instruments).

In existing medical instrumentation cases that provide brackets to hold medical instruments, the brackets or instrument holding mechanisms are often fixed in place or can be provided from a molded assembly indistinguishable from the instrument case. The brackets are generally fitted to a specific set of tools that is defined by a manufacturer-specified configuration. Existing medical instrumentation cases can also provide a silkscreened label or indication printed directly on the case to show which tool fits in which bracket. Problems are caused, then, when a doctor or other interested party chooses to substitute or remove tools from the manufacturer-specified set of instruments. This results in brackets not being used for their intended purposes, and loose instruments being kept and transported in the tray. In particular, this may cause a loss or mixup of instrumentation, and incomplete or improper cleaning, when a tray having loose and unsecured instruments is placed in an automated cleaning machine or sterilizer.

In one example further disclosed herein, a modular bracket is provided for a custom placement and fit with a specific tool, device, or other instrument associated with a medical procedure. The modular bracket can be placed in a modular instrument case assembly that can include one or more modular brackets to receive an instrument, one or more platforms to facilitate modular placement of the modular brackets, one or more trays to provide the platforms, and a housing which can receive the trays (and the associated platforms and modular brackets). The platforms, housing, and trays of the modular instrument case assembly can provide a sterilizable case for storage, cleaning, or transport of the instrumentation held by the modular brackets.

As a further example, the modular bracket can be used in connection with an instrument case that provides a platform having a grid pattern of apertures with a modular mounting surface. This modular mounting surface can be provided by rows of holes for custom placement of the modular brackets in respective fastening receptacles at any location that lines up with the holes or other apertures. One or more removable or integrated fasteners can be used to couple the modular bracket with the fastening receptacles provided by the grid pattern, resulting in a large number of possible locations or placements for the modular bracket on the platform.

Accordingly, the example modular brackets described herein can be positioned, repositioned, substituted, and removed from a medical instrumentation case in a quick and modular fashion. The modular brackets, however, can be specific to the instrumentation and provide an indication of the specific instrumentation that is received by the modular bracket. In contrast to existing instrumentation case brackets, use of the modular bracket and modular instrument case assembly does not secure a tool or bracket to a fixed, pre-printed location. Thus, use of a modular bracket in a medical instrumentation setting enables a doctor to choose which set of tools (and which set of modular brackets) to include in his or her customized instrumentation case, and add, remove, or replace specific brackets to adapt to the specific procedure or arrangement of instrumentation that is desired.

The example modular brackets described herein can provide a user-visible indication of the received instrument directly on, within, or from the modular bracket. This indication can indicate to a user which instrument fits and is appropriate for placement into the modular bracket. This is in contrast to existing case designs where a permanent marking in a specific language is placed in the case next to a permanent bracket, or case designs that allow movable generic brackets but that do not provide a specific structure to securely hold particular instrumentation.

The indication of the particular instrument that fits into the modular bracket can be provided through the shape, structure, or other visible characteristic of the modular bracket, such as an outside perimeter (and in some cases a corresponding interior perimeter) defining an outline of the modular bracket shape that resembles the instrument. The indication can also be provided separately or in combination with other forms specific to the modular bracket, including a fixed or removable label with alphanumeric text (in the form of user-readable words or an item number, for example), an icon, a color indication, shapes or symbols, and like user-perceptible indications. As another example, the color indication of the modular bracket can match a particular tool in a color-unique tool set, or identifiable color markings provided on a particular tool. In some examples, the color may be covered when the instrument is received by the modular bracket.

The indication that is provided by the shape, structure, or other visible characteristic of the modular bracket can be specific to an unused, used, assembled, or disassembled state of an instrument. For example, a modular bracket can be shaped to generally resemble the shape of a specific tool when fully disassembled and placed in a mode for cleaning. As another example, the modular bracket can be shaped to only hold an instrument prior to its use, when in an unused or in a clean state, to prevent placement of a non-reusable or dirty instrument in a specific modular bracket. Additional examples of the design, structure, and use of the modular brackets are illustrated in the accompanying drawings described herein.

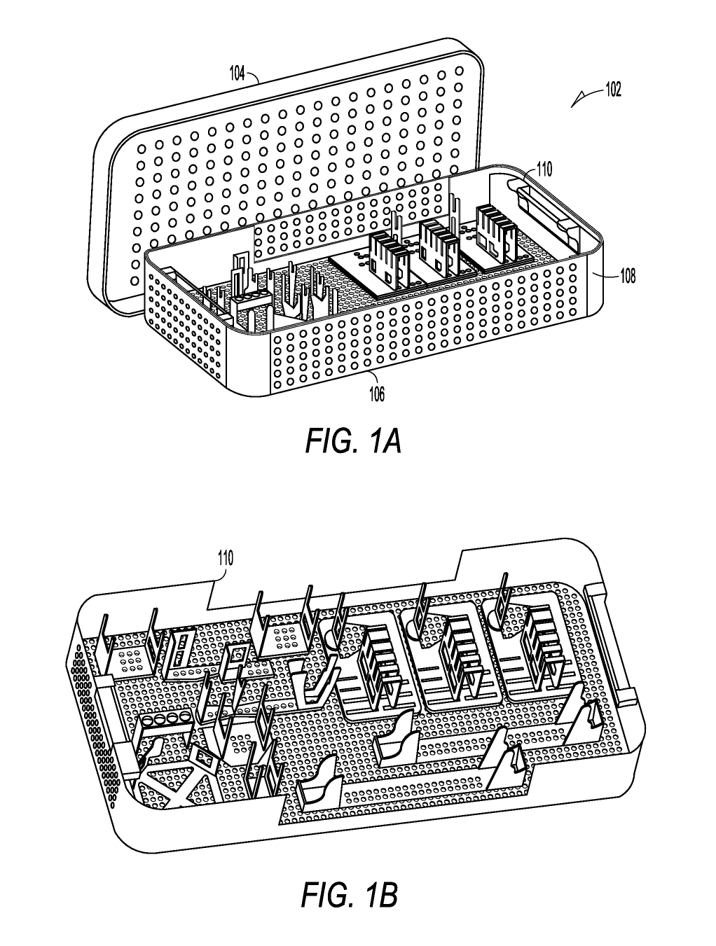

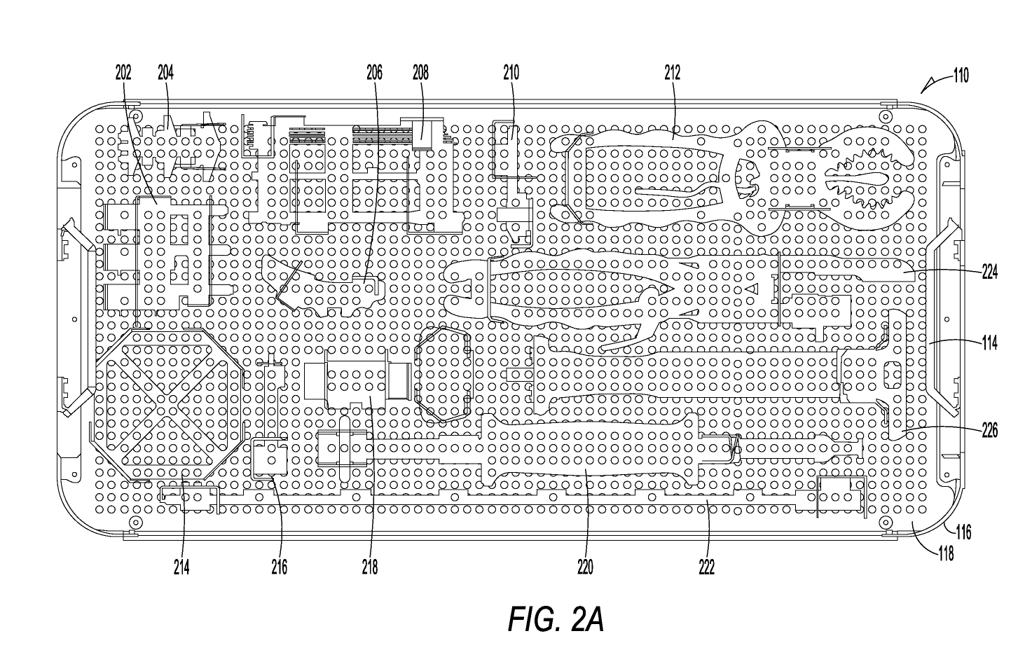

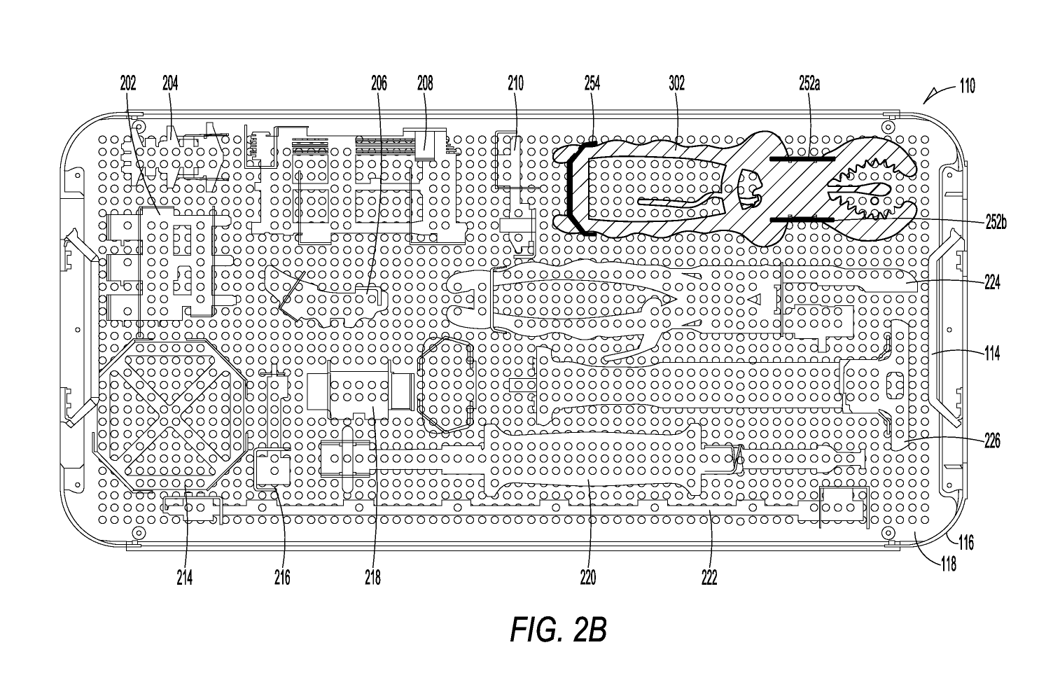

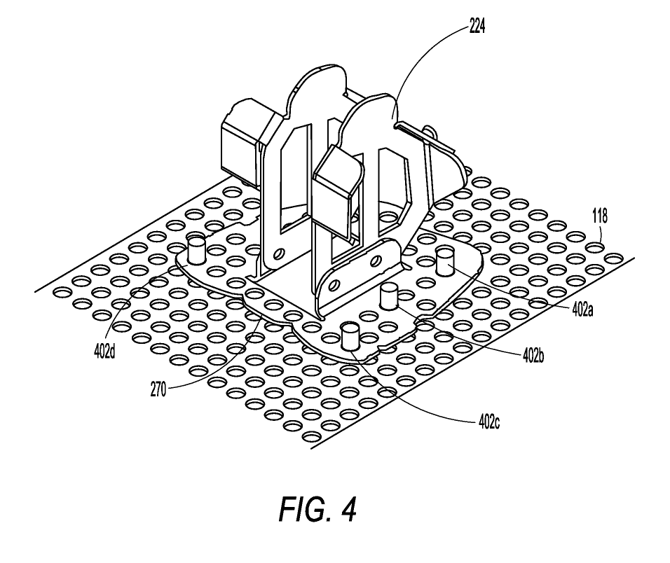

FIG. 1A provides an illustration of a modular instrument case assembly 102 providing a configuration of a plurality of modular brackets to hold instruments within an instrument tray 110 according to one example. The instrument case assembly 102 includes a housing provided by a container 108 and a lid 104. The lid 104 is shown as being removable from the container 108, but the lid 104 can be permanently coupled to the container 108 in some configurations. The housing is shaped to fit one or more instrument case trays within the container 108, including the instrument tray 110. The instrument tray 110 provides various apertures for receipt and placement of a plurality of modular brackets. Examples of an instrument tray are shown in more detail in FIG. 1B and FIG. 2 and described below, and the attachment between a modular bracket and a platform provided by an instrument tray is shown in more detail in FIG. 4 and described below.

The lid 104, the container 108, and the instrument tray 110 of the instrument case assembly 102 are illustrated as providing a plurality of apertures arranged in a “grid”-like pattern of circular holes 106. These apertures can also serve as ventilation holes, mounting holes, or cleaning holes. The apertures within the lid 104, the container 108, or the instrument tray 110 can be provided by non-circular hole shapes, such as a rectangular shape, a square shape, a cruciform shape, a slot shape, or various combinations of geometrical shapes. The instrument tray 110 can be removed from the housing of the instrument case assembly 102 for access to specific instrumentation or other trays held in the container 108, or for specific operations such as for placement in an automated cleaning or sterilization machine.

FIG. 1B provides an illustration of a modular instrument case assembly according to another example. FIG. 1B specifically illustrates a perspective view of the instrument tray 110 providing a configuration of modular brackets for receiving multiple instruments within the instrument tray 110. Within the instrument tray 110, the various modular brackets are mounted to a platform of the instrument tray 110 providing attachment receptacles. These attachment receptacles can be provided by apertures of the instrument tray 110, arranged in a pattern and shaped in a circular or other geometrical shape. An attachment member (e.g., an attachment platform or base) of a modular bracket can be mounted to the attachment receptacle of the instrument tray 110 using a non-permanent fastener such as a pin, peg, screw, bolt, and the like. In some examples, the fastener may be integrated onto a portion of the attachment member or the associated modular bracket.

The instrument tray 110 can provide a coupling mechanism to secure attachment with an instrument tray cover (not shown), and the instrument tray case 110 can also provide a handle or other mechanism to facilitate user or machine handling. The size and structure of the instrument tray 110 and an instrument tray cover can be adapted for placement in automated cleaning, decontamination, or sterilization machines, or storage racks and facilities. For example, a series of perforations or holes can be exposed on each side of the instrument tray 110 and the instrument tray cover for washing and cleaning of the instruments in the instrument tray 110. The instrument tray 110 can be provided from metal (such as stainless steel), plastic, or other rigid material suitable for cleaning, transport, storage of medical instrumentation.

FIG. 2A illustrates a top view of modular brackets placed within the instrument tray 110, the modular brackets being placed to receive and hold various instruments in a stationary placement according to an example. The instrument tray 110 includes a handle 114 to facilitate carrying of the tray. The instrument tray 110 includes a side wall 116 that is perpendicular to a tray platform 118. The side wall 116 can extend vertically above each of the brackets and instruments placed within the instrument tray 110. The side wall 116 can be shaped to receive or couple to an instrument tray cover, or for placement in the instrument case assembly 102.

The tray platform 118 can provide a plurality of apertures arranged in a grid, to facilitate mounting and attachment of modular brackets. The aperture openings can also provide functionality of a sterile instrumentation case, providing openings for purposes such as cleaning, sterilization, and storage.

A variety of modular brackets are illustrated as being mounted to the tray platform 118, with each modular bracket conforming to specific instrumentation components. The modular brackets specifically conform to a variety of shapes and sizes of the instrumentation components, as evidenced by the disparate shapes of modular brackets 202, 204, 206, 208, 214, 216, 218, 220, 222, 224, 226. Some of these modular brackets include various support members extending from the tray platform 118 that are structured to receive an instrument, hold the instrument at a fixed position upon receipt, and maintain an engagement with the instrument.

FIG. 2B, similar to FIG. 2A, illustrates a top view of the modular brackets 202, 204, 206, 208, 214, 216, 218, 220, 222, 224, 226 placed within the instrument tray 110 to receive various instruments, with placement of an instrument 302 into the modular bracket 212 (not visible in FIG. 2B) according to an example. The instrument 302 is held within the instrument tray 110 using the support members 252a, 252b, 254 that extend perpendicular to the attachment member (e.g., the base) of the modular bracket 212.

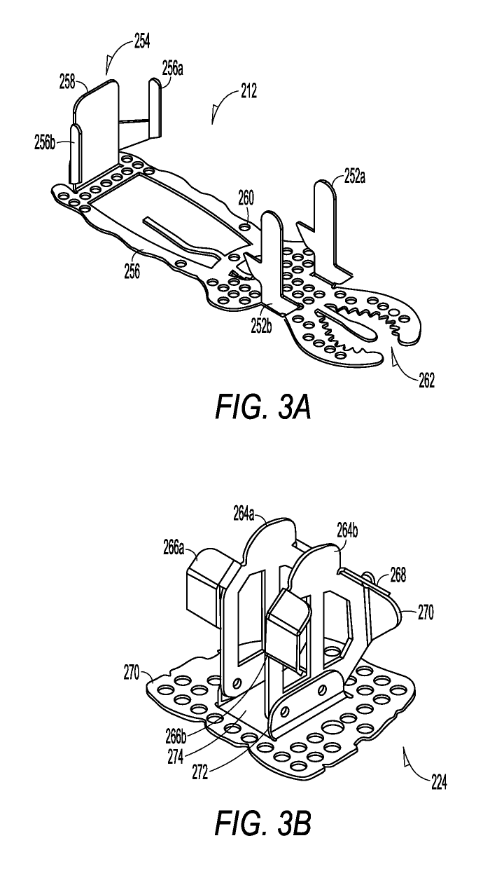

FIG. 3A provides a close-up perspective view of the modular bracket 212 detached from an instrument tray case and platform. FIG. 3A illustrates the structures of the modular bracket 212, including the attachment member 256 and the support members 254, 252a, 252b. The attachment member 256 is structured for attachment to a platform of a medical instrument tray or other suitable mounting structure using one or more coupling or attachment mechanisms. As one example, a fastener can be used to couple the attachment member 256 to a medical instrument tray by placement of the fastener in an aperture 260. The attachment member 256 provides a plurality of apertures that generally align with the mounting surface of a platform of a medical instrument tray. A suitable fastener can be attached to only select apertures and need not be attached to all apertures provided by the attachment member 256.

As further illustrated in FIG. 3A, a plurality of support members are provided with various shapes to hold, engage, and secure the specific instrument in a fixed position with respect to the mounting surface of the platform or other structure of the medical instrumentation tray. The support members 254, 252a, 252b can be used to couple with specific portions of the instrument above the platform, such as to interlock or engage with specific sides of the instrument. The support members can directly or indirectly connect to the attachment member 256, for example as depicted by the support member 254 providing a central support member 258 directly connected to the attachment member 256, with the support members 256a and 256b directly connecting to the central support member 258. The support members 256a and 256b are provided as integral parts of the overall modular bracket 212, and are directly connected to the attachment member 256 so as to allow the entire assembly of the modular bracket 212 to move as a single unit.

As also illustrated in FIG. 3A, the shape of the modular bracket 212 provides a visible indication of the specific instrumentation received by the modular bracket 212, allow a user to easily identify the specific instrumentation received by the support member(s) of the modular bracket 212. Specifically the outline 262 of the modular bracket 212 is designed to resemble the specific instrument held by the modular bracket. The perimeter of modular bracket 212 (including detail such as the teeth in the outline 262) match a specific tool.

FIG. 3B provides another close-up perspective view of a modular bracket 228 shaped to provide engageable mating with another instrument according to an example. The modular bracket 228 includes an attachment member 270 with apertures of multiple sizes. For example, certain sets of apertures can be used to align with one configuration of a mounting surface (e.g., a specific instrument tray from a vendor or manufacturer), while other sets of apertures can be used to align with another configuration of a mounting surface (e.g., a specific instrument tray from another vendor or manufacturer).

FIG. 3B also provides an illustration of a modular bracket 224 having a plurality of support members 264a, 264b, coupled to support member base 274, the modular bracket 224 used for receiving and engaging with various instrumentation or instrumentation pieces. The modular bracket provides a plurality of coupling members extending perpendicular from the support members 264a to couple with and secure portions of the instrumentation, including coupling members 266a, 266b, 268, and 270.

The modular bracket can be shaped, structured, or used to facilitate the management of multiple instrumentation pieces and units. For example, a single modular bracket can be shaped to hold more than one instrument at the same time. Likewise, a single modular bracket can be shaped to hold multiple pieces of an instrument. As is also apparent, multiple modular brackets can be used to hold a single instrument or instrumentation piece. The modular bracket and the components of the modular bracket may be constructed from one or multiple types of materials, including metal, plastic, silicone, and the like.

FIG. 4 illustrates a close-up perspective view of a fastener coupling between the modular bracket 224 and an instrument tray platform 118. As shown, the attachment member 270 facilitates the connection of a number of fasteners in a number of fastener receptacles provided by the instrument tray platform 118. These fasteners include spring-loaded pins 402a, 402b, 402c, and 402d, placed among various locations of the modular bracket 224 to provide a secure placement and alignment between the apertures of the modular bracket 224 and the instrument tray platform 118.

The modular brackets can be coupled to the instrumentation case using any number of techniques. For example, the modular brackets can be removably coupled to the instrumentation case using quick-fastener pins, which can be attached to and removed from the case by any number of manual or tool-assisted techniques. For example, spring loaded pins can be used to quickly attach the modular brackets to the attachment receptacles of an instrumentation case by hand without use of tools.

Although the present structures were described with reference to the placement and use of medical instruments and tools in a storage case, it will be understood that the applicability of the identified structures, configurations, and techniques extends to other types of instruments and use cases, including uses in non-medical settings. Modifications can be made to the various case and bracket structures described herein that provide the modular functionality and indications.

The above detailed description includes references to the accompanying drawings, which form a part of the detailed description. The drawings show, by way of illustration, specific examples in which the invention can be practiced. These examples are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls. In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other examples can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed example. Thus, the following claims are hereby incorporated into the Detailed Description as examples or embodiments, with each claim standing on its own as a separate example, and it is contemplated that such examples can be combined with each other in various combinations or permutations. The scope of the invention should be determined with reference to the appended claims, along with the full scope of equivalents to which such claims are entitled.

1. A modular bracket, comprising:
an attachment member structured to facilitate a releasable attachment to a mounting surface of a medical instrumentation tray, wherein the attachment member is configured for modular attachment of the modular bracket to the medical instrumentation tray at a plurality of locations on the mounting surface; and
a support member structured to receive a particular instrument, wherein the support member is configured to hold the particular instrument at a fixed position with respect to the mounting surface upon receipt of the particular instrument by the support member;
wherein the modular bracket is structured to provide a visual indication of the particular instrument, to allow user identification of the particular instrument received by the support member.

2. The modular bracket of claim 1, wherein the visual indication is provided by the shape of the attachment member, and wherein an outline of the attachment member corresponds to a representation of a shape of the particular instrument.

3. The modular bracket of claim 1, wherein the visual indication is provided by at least one color of the modular bracket, and wherein the at least one color of the modular bracket corresponds to at least one color of the particular instrument.

4. The modular bracket of claim 1, wherein the visual indication is provided by alphanumeric text, affixed to the modular bracket, that corresponds to the particular instrument.

5. The modular bracket of claim 1, further comprising at least one additional support member.

6. The modular bracket of claim 5, wherein the at least one additional support member is structured to receive one or more additional particular instruments.

7. The modular bracket of claim 6, wherein the modular bracket is structured to hold separate portions of the particular instrument in the support member and the additional support member upon disassembly of the particular instrument into the separate portions.

8. The modular bracket of claim 1, wherein the modular bracket is structured to receive a portion of the particular instrument, and wherein at least one additional modular bracket configured for modular attachment to the medical instrumentation tray is shaped to receive another portion of the particular instrument.

9. The modular bracket of claim 1, wherein the attachment member includes one or more attachment receptacles configured to receive one or more fasteners.

10. The modular bracket of claim 1, wherein the particular instrument is an instrument used in an orthopedic procedure, and wherein the medical instrumentation tray provides modular attachment locations for a plurality of modular brackets including the modular bracket, the plurality of modular brackets configured for holding respective particular instruments used in the orthopedic procedure.

11. A modular bracket, comprising:
an attachment member structured to facilitate a releasable attachment to a mounting surface of a medical instrumentation tray, wherein the attachment member is configured for modular attachment of the modular bracket to the medical instrumentation tray at one of a plurality of locations on the mounting surface, wherein the attachment member provides a plurality of apertures to receive a plurality of fasteners for attachment with the mounting surface; and
a support member structured to receive a particular medical instrument, wherein the support member is configured to hold the particular medical instrument at a fixed position with respect to the mounting surface upon receipt of the particular instrument by the support member;
wherein the modular bracket is structured to provide a visual indication of the particular instrument by the shape of the attachment member, to allow user identification of the particular instrument received by the support member, wherein an outline of the attachment member corresponds to a representation of a shape of the particular medical instrument; and
wherein the medical instrumentation tray provides for the releasable attachment with the modular bracket at plurality of apertures of the mounting surface using the plurality of fasteners upon alignment with the plurality of apertures of the attachment member.

12. A modular instrument case assembly, comprising:
a platform structured for modular placement of one or more modular brackets using one or more removable fasteners; and
a modular bracket structured for removable coupling to one of a plurality of positions on the platform, the modular bracket having an attachment member structured to facilitate a releasable attachment to the platform, and a support member structured to receive and hold a particular instrument at a fixed position with respect to the platform;
wherein the modular bracket provides a visual indication of the particular instrument received by the support member.

13. The modular instrument case assembly of claim 12, wherein the platform includes a plurality of platform apertures arranged according to a pattern and the modular bracket includes at least one bracket aperture, wherein alignment of the at least one bracket aperture with one of the platform apertures forms a fastening receptacle configured to receive a fastener for securing the modular bracket to the platform.

14. The modular instrument case assembly of claim 13, wherein the fastener includes one or more of: a peg, a screw, a bolt, or a spring-loaded pin.

15. The modular instrument case assembly of claim 13, wherein a perimeter of the modular bracket is shaped to provide an indication of a particular instrument to be received by the modular bracket.

16. The modular instrument case assembly of claim 12, wherein the modular bracket comprises a metallic or plastic bracket.

17. The modular instrument case assembly of claim 12, wherein the modular bracket comprises at least two colors, wherein one of the colors is visible when the instrument is detached from the modular bracket and is covered when the instrument is received by the modular bracket.

18. The modular instrument case assembly of claim 12, wherein the platform is provided by a medical instrumentation tray.

19. The modular instrument case assembly of claim 12, further comprising:
a medical instrumentation housing; and
a medical instrumentation tray;
wherein the platform is included in the medical instrumentation tray, and wherein the medical instrumentation tray is shaped for placement in the housing of the modular instrument case assembly.

20. The modular instrument case assembly of claim 19, further comprising:
a plurality of additional modular brackets; and
a plurality of orthopedic instruments placed among the modular bracket and the plurality of additional modular brackets.
